SEQUENCE LISTING (4 sequences)

SEQ ID NO: 1 - DNA, 23 bp, Cordyceps militaris
  source 1..23: mol_type = other DNA
atggaacacagatcgagcgacac

SEQ ID NO: 2 - DNA, 25 bp, Cordyceps militaris
  source 1..25: mol_type = other DNA
atataccttcgcgatcattgcccag

SEQ ID NO: 3 - DNA, 22 bp, Cordyceps militaris
  source 1..22: mol_type = other DNA
tgttttgtcgcgatggttctgg

SEQ ID NO: 4 - DNA, 22 bp, Cordyceps militaris
  source 1..22: mol_type = other DNA
cctctggaggttctgcattcca

(19) United States
(12) Patent Application Publication
(10) Pub. No.: US 2026/0061015 A1
(43) Pub. Date: Mar. 5, 2026

(54) Cordyceps Militaris Designated as Strain M2-116-04 Compositions, Methods of Making, Methods of Using, and Methods for Treating a Mammal

(71) Applicant: M2 Ingredients, Inc., Vista, CA (US)

(72) Inventors: Samuel Shandor Andrasko, Lakeside, CA (US); Julie Daoust, Carlsbad, CA (US)

(21) Appl. No.: 19/212,318

(22) Filed: May 19, 2025

Related U.S. Application Data
(63) Continuation-in-part of application No. 19/096,553, filed on Mar. 31, 2025.
(60) Provisional application No. 63/636,605, filed on Apr. 19, 2024.
(60) Provisional application No. 63/650,718, filed on May 22, 2024.

(51) Int. Cl. A61K 36/068; A01G 18/00; A01H 15/00; A61P 1/00

(52) U.S. Cl. CPC A61K 36/068; A01G 18/00; A01H 15/00; A61P 1/00

(57) ABSTRACT

Embodiments include a not naturally occurring strain of Cordyceps militaris fungus designated as strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802. The isolated mycelium is characterized by a phenotype having an abundance of sporocarps on top of a substrate and/or along a parameter of a bioreactor bag, and also characterized by a phenotype having fruiting initiation at or near 18 days after inoculation, a phenotype having sporocarps maturing at or near 55 days after inoculation, and/or a phenotype having a 10-to-15-day colonization time on hulled oats. Applications include functional foods, prebiotic formulations, and immune support products, with the strain available in powder, capsule, or drink mix forms.

### CROSS REFERENCE TO RELATED APPLICATIONS

The present U.S. Non-Provisional Patent application claims the priority benefit of U.S. Provisional Patent Application Ser. No. 63/650,718 filed on May 22, 2024, and titled “Cordyceps militaris Designated as Strain M2-116-04”. The present U.S. Non-Provisional Patent Application is a continuation-in-part of U.S. patent application Ser. No. 19/096,553 filed on Mar. 31, 2025, and titled “Systems and Methods of Liquid Submerged Fermentation Using Liquid Dilution Spawning for Expanding Inoculum of Fungal Species”, which claims the priority benefit of U.S. Provisional Patent Application Ser. No. 63/636,605 filed on Apr. 19, 2024, and titled “Systems and Methods of Liquid Submerged Fermentation for Expanding Inoculum of Fungal Species”. All of the aforementioned disclosures are hereby incorporated by reference herein in their entireties, including all patent deposit material(s) and all references cited therein.

### REFERENCE TO SEQUENCE LISTINGS

The present application is filed with sequence listing(s) attached hereto and incorporated by reference, including paragraphs through of the specification as filed.

### REFERENCE TO AN ELECTRONIC SEQUENCE LISTING

The contents of the electronic sequence listing (M2INGR Primers.xml; Size: 4,656 bytes; and Date of Creation: Sep. 17, 2025) is herein incorporated by reference in its entirety.

### FIELD OF THE TECHNOLOGY

The present disclosure pertains to biotechnology, specifically to microbial technology and functional foods, with a focus of some embodiments on the cultivation, composition, and applications of Cordyceps militaris fungus designated as strain M2-116-04.

### BACKGROUND

The approaches described in this section could be pursued, but are not necessarily approaches that have previously been conceived or pursued. Therefore, unless otherwise indicated, it should not be assumed that any of the approaches described in this section qualify as prior art merely by virtue of their inclusion in this section.

Advances in functional foods have driven significant interest in natural ingredients that can beneficially modulate gut microbial communities and metabolic function. Traditional medicinal mushrooms have been used for centuries in various cultures for their health-promoting properties, including immunomodulation, anti-inflammatory activity, and support for overall wellness. Recent research has indicated that components found in mushrooms can influence the profile of short-chain fatty acids (SCFAs) produced in the colon, which are important for maintaining gut barrier integrity, balancing immune responses, and regulating energy metabolism. However, many existing products rely on highly processed extracts or isolated polysaccharide fractions that may not capture the complete spectrum of bioactive compounds naturally present in unprocessed mushrooms.

Despite promising effects demonstrated with isolated components, challenges remain in obtaining reproducible and synergistic benefits when only a fraction of the mushroom is used. Conventional processing techniques can alter the chemical composition and attenuate the potential for complementary interactions among constituents such as fungal mycelium, fruiting body, and other parts. Moreover, established in vitro models and fermentation assays used to evaluate prebiotic effects often do not fully capture the complex interplay between diverse mushroom-derived metabolites and the gut microbiota. Such limitations underscore the need for innovations that utilize minimally processed, full-spectrum mushroom formulations. These novel approaches aim to preserve the diverse array of biomolecules that support a beneficial fermentation profile, robust SCFA production, and provide enhanced support for intestinal health and immune function.

### BRIEF SUMMARY OF EXEMPLARY EMBODIMENTS

Cordyceps militaris is difficult to cultivate on a large scale. Additionally, the sporocarp of Cordyceps militaris are relatively small, which makes them time-consuming to harvest. The sporocarp is also known as fruiting body, fruit body or fruitbody. The amount of active compounds in Cordyceps militaris can vary depending on the growing conditions, the extraction process, and the storage conditions. Consistency is key. The exemplary embodiments herein solve these problems. For example, Cordyceps militaris fungus designated as strain M2-116-04 is not naturally occurring and is the product of human intervention that includes increased cordycepin (C) content in the sporocarp and biomass compared with other strains. Significantly, Cordyceps militaris fungus designated as strain M2-116-04 has markedly different characteristics than naturally occurring strains.

In some embodiments the techniques described herein relate to a not naturally occurring Cordyceps militaris fungus designated as strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802. Significantly, Cordyceps militaris fungus designated as strain M2-116-04 has markedly distinctive characteristics than naturally occurring strains.

In some embodiments, the techniques described herein relate to a not naturally occurring Cordyceps militaris fungus designated as strain M2-116-04, characterized by a phenotype having an abundance of sporocarps on top of a substrate and along a parameter of a bioreactor bag.

In some embodiments, the techniques described herein relate to a not naturally occurring Cordyceps militaris fungus designated as strain M2-116-04, characterized by a phenotype having fruiting initiation at or near 18 days after inoculation.

In some embodiments, the techniques described herein relate to a not naturally occurring Cordyceps militaris fungus designated as strain M2-116-04, characterized by a phenotype having sporocarps maturing at or near 55 days after inoculation.

In some embodiments, the techniques described herein relate to a not naturally occurring Cordyceps militaris fungus designated as strain M2-116-04, characterized by a phenotype having a 10-to-15-day colonization time on hulled oats.

In some embodiments, the techniques described herein relate to a not naturally occurring Cordyceps militaris fungus designated as strain M2-116-04, characterized by a phenotype optimized to grow on oats.

In some embodiments, the techniques described herein relate to a not naturally occurring Cordyceps militaris fungus designated as strain M2-116-04, characterized by a phenotype having a beta-glucan content of approximately 20% to 25% as represented as a percentage of a total mycelium and sporocarps.

In some embodiments, the techniques described herein relate to a not naturally occurring Cordyceps militaris fungus designated as strain M2-116-04, characterized by a phenotype having a beta-glucan content of approximately 26% to 31% as represented as a percentage of a total mycelium and sporocarps.

In some embodiments, the techniques described herein relate to a not naturally occurring Cordyceps militaris fungus designated as strain M2-116-04, characterized by a phenotype having a beta-glucan content of approximately 32% to 37% as represented as a percentage of a total mycelium and sporocarps.

In some embodiments, the techniques described herein relate to a not naturally occurring Cordyceps militaris fungus designated as strain M2-116-04, characterized by a phenotype having a beta-glucan content of approximately 38% to 43% as represented as a percentage of a total mycelium and sporocarps.

In some embodiments, the techniques described herein relate to a not naturally occurring Cordyceps militaris fungus designated as strain M2-116-04, characterized by a phenotype having a beta-glucan content of approximately 44% to 49% as represented as a percentage of a total mycelium and sporocarps.

In some embodiments, the techniques described herein relate to a not naturally occurring Cordyceps militaris fungus designated as strain M2-116-04, characterized by a phenotype having a cordycepin content of approximately 0.4 milligrams per gram to 1.1 milligrams per gram as represented relative to a total mycelium and sporocarps.

In some embodiments, the techniques described herein relate to a not naturally occurring Cordyceps militaris fungus designated as strain M2-116-04, in a powder form.

In some embodiments, the techniques described herein relate to a not naturally occurring Cordyceps militaris fungus designated as strain M2-116-04, in a capsule form.

In some embodiments, the techniques described herein relate to a not naturally occurring Cordyceps militaris fungus designated as strain M2-116-04, in a drink mix form for cold and hot beverages applications.

In some embodiments, the techniques described herein relate to a method for treating a mammal to improve immune functioning of the mammal, the method including administering an effective dose of Cordyceps militaris mushroom powder including Cordyceps militaris mycelium and sporocarps.

In some embodiments, the techniques described herein relate to a method, wherein the effective dose is administered once every 12 days over 8 separate consecutive 12-day intervals.

In some embodiments, the techniques described herein relate to a method, wherein the Cordyceps militaris mushroom powder includes not naturally occurring Cordyceps militaris fungus designated as strain M2-116-04.

In some embodiments, the techniques described herein relate to a method, further including the effective dose being 500 mg of the Cordyceps militaris mushroom powder per kg of body weight of the mammal.

In some embodiments, the techniques described herein relate to a method, further including the effective dose being 1000 mg of the Cordyceps militaris mushroom powder per kg of body weight of the mammal.

In some embodiments, the techniques described herein relate to a method, further including the effective dose being 2000 mg of the Cordyceps militaris mushroom powder per kg of body weight of the mammal.

In some embodiments, the techniques described herein relate to a method, further including the mammal being a human.

In some embodiments, the techniques described herein relate to a method for treating a mammal to improve gut functioning of the mammal, the method including administering an effective dose of Cordyceps militaris mushroom powder including not naturally occurring Cordyceps militaris fungus designated as strain M2-116-04, the mushroom powder including Cordyceps militaris mycelium and sporocarps and being a prebiotic.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee. With respect to FIG. 5 and FIG. 9, the color drawings are necessary as the only practical medium by which aspects of the claimed subject matter may be accurately conveyed. The color of Cordyceps militaris may vary. Thus, the color drawings help differentiate the not naturally occurring strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802 from other strains of Cordyceps militaris. The color drawings also help differentiate a stage of growth of not naturally occurring strain M2-116-04. Significantly, the color drawings also help show the markedly distinctive characteristics of Cordyceps militaris fungus designated as strain M2-116-04 compared to naturally occurring strains.

While this technology is susceptible of embodiment in many different forms, there is illustrated in the drawings, and will herein be described in detail, several specific embodiments with the understanding that the present disclosure is to be considered as an exemplification of the principles of the technology and is not intended to limit the technology to the embodiments illustrated.

It will be understood that like or analogous elements and/or components, referred to herein, may be identified throughout the drawings with like reference characters. It is further understood that several of the figures are merely schematic representations of the present technology. As such, some of the components may be distorted from their actual scale for pictorial clarity.

FIG. 1 is a flowchart illustrating an exemplary method for propagating a Cordyceps militaris fungus, according to some embodiments of the present disclosure.

FIG. 2 is a drawing of an overview of the selection by breeding and strain selection to produce the improved and not naturally occurring Cordyceps militaris designated as strain M2-116-04, according to some embodiments of the present disclosure.

FIG. 3 is a drawing of an agar plate culturing Cordyceps militaris for selecting improved, not naturally occurring strains, according to various embodiments of the present disclosure.

FIG. 4 is a drawing of an agar plate culturing Cordyceps militaris for selecting improved, not naturally occurring strains including a small section of the overlapping mycelium cut, according to various embodiments of the present disclosure.

FIG. 5 is a color drawing a fully colonized and fully fruited one pound block of not naturally occurring Cordyceps militaris fungus designated as strain M2-116-04, according to various embodiments of the present disclosure.

FIG. 6 is a table showing the trending of cordycepin (C) content in sporocarps (e.g., fruiting bodies) and biomass ordered by linear coverage of the sporocarps (e.g., fruits) for various strains of Cordyceps militaris fungus including strain M2-116-04, according to various embodiments of the present disclosure.

FIG. 7 is a table showing cordycepin (C) in Cordyceps militaris fungus designated as strain M2-116-04 per cubic foot of grow space using tray culture compared with liquid bag culture, according to various embodiment of the present disclosure.

FIG. 8 is a drawing of an overview of the selection by breeding and strain selection including parameters and potential to produce the improved, not naturally occurring Cordyceps militaris designated as strain M2-116-04, according to some embodiments of the present disclosure.

FIG. 9 is a color drawing showing a phenotype description of Cordyceps militaris fungus designated as strain M2-116-04, according to embodiments of the present disclosure.

### DETAILED DESCRIPTION OF EXEMPLARY EMBODIMENTS

The present disclosure relates generally to the field of biotechnology, with a particular focus on microbial technology, functional foods, and the cultivation and applications of Cordyceps militaris fungus, specifically the not naturally occurring strain designated as M2-116-04. The subject matter encompasses compositions, methods of making, and methods of using this strain, including potential applications in health, nutrition, and industrial processes. The described embodiments aim to address challenges in large-scale cultivation, consistency in bioactive compound production, and the optimization of fungal growth conditions to enhance functional properties.

The not naturally occurring Cordyceps militaris fungus designated as strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802.

The examples and embodiments described herein are provided for illustrative purposes only and are not intended to limit the scope of the subject matter. Certain details, well-known to those skilled in the art, may be omitted for clarity and brevity. Furthermore, various modifications, substitutions, and rearrangements of the described elements and methods may be made without departing from the spirit and scope of the subject matter, as defined by the appended claims. The cultivation and utilization of Cordyceps militaris fungus have long been recognized for their potential health benefits, including immune modulation, anti-inflammatory properties, and support for overall wellness. However, conventional approaches to cultivating Cordyceps militaris face significant challenges. These include inconsistent production of bioactive compounds such as cordycepin and beta-glucans, difficulty in large-scale cultivation due to the small size of sporocarps, and variability in growth conditions that impact the reproducibility of desired phenotypes. Additionally, traditional methods often rely on highly processed extracts or isolated polysaccharide fractions, which fail to capture the full spectrum of bioactive compounds naturally present in the fungus. This limits the synergistic effects of the mycelium, fruiting body, and other fungal components, reducing the overall efficacy of the product.

In various embodiments, the present technology addresses these limitations by introducing a novel not naturally occurring strain of Cordyceps militaris, designated as strain M2-116-04, which has been optimized for large-scale cultivation and enhanced production of bioactive compounds. This not naturally occurring strain exhibits several improved phenotypic characteristics, including faster colonization times, fruiting initiation at or near 18 days after inoculation, and sporocarps maturing at or near 55 days after inoculation. Furthermore, strain M2-116-04 demonstrates increased beta-glucan content, ranging from approximately 20% to 49%, and cordycepin content of approximately 0.4 to 1.1 milligrams per gram, as represented relative to the total mycelium and sporocarps. These enhancements ensure consistent production of bioactive compounds, addressing the variability seen in conventional strains.

In various embodiments, the present technology also incorporates advanced methods for propagating the strain, including liquid submerged fermentation techniques and optimized bioreactor systems. These methods enable efficient inoculation and growth on organic grain substrates, such as oats, which further enhance the nutritional profile and reduce contamination risks. The liquid fermentation process ensures uniform dispersion of mycelium fragments, leading to increased fungal biomass and full-spectrum fungal production. This approach not only improves the scalability of cultivation but also preserves the diverse array of biomolecules necessary for promoting synergistic health benefits.

By leveraging these advancements, the present technology provides a robust solution to the challenges of cultivating Cordyceps militaris at scale while maintaining elevated levels of bioactive compounds. This enables the production of minimally processed, full-spectrum fungal formulations that offer enhanced prebiotic activity, immune support, and overall health benefits, surpassing the limitations of conventional methods and products.

According to various exemplary embodiments, an organic grain substrate is a sterile, nutrient-rich medium used to grow Cordyceps militaris. It may be composed of a combination of whole grains, such as rice, rye, oats, or wheat, supplemented with other organic ingredients like wheat bran, brewer's yeast, and calcium carbonate. These substrates provide the necessary nutrients and support for the mycelium, the vegetative part of the Cordyceps militaris, to grow and develop into sporocarps.

In various exemplary embodiments, organic grain substrates are preferred over non-organic substrates due to several advantages, including reduced risk of contamination. Organic grains are less likely to contain harmful chemicals or pathogens that could contaminate the Cordyceps militaris. They also have enhanced nutritional value. Organic grains are generally higher in nutrients and minerals, which can contribute to the nutritional value of the Cordyceps militaris grown on them.

Additionally, in certain exemplary embodiments, the substrate should be sterilized to eliminate any potential contaminants before inoculating the substrate with Cordyceps militaris spawn. The substrate should have a balanced moisture content to support optimal mycelium growth and comprise a balanced blend of nutrients to provide the Cordyceps militaris with essential elements for healthy development.

In exemplary embodiments, the whole mycelium may be characterized by a phenotype having a cordycepin content of approximately 0.4 milligrams per gram to 1.1 milligrams per gram as represented relative to the total mycelium and sporocarps. The whole mycelium, in other exemplary embodiments, may be characterized by a phenotype having a cordycepin content of approximately 0.4 milligrams per gram to 0.75 milligrams per gram as represented relative to the total mycelium and sporocarps, or the whole mycelium may be characterized by a phenotype having a cordycepin content of approximately 0.75 milligrams per gram to 1.1 milligrams per gram as represented relative to the total mycelium and sporocarps in other exemplary embodiments.

According to various exemplary embodiments, beta-glucan, is a type of soluble fiber found in the cell walls of Cordyceps militaris. Beta-glucan is a natural compound with a wide range of potential health benefits. Beta-glucan has been shown to have a number of potential health benefits, including immune system support. Beta-glucan is a immunomodulator meaning it can help to support the immune system's ability to fight off infection. Beta-glucan does this by activating certain immune cells, such as macrophages and natural killer cells. Beta-glucan is a prebiotic, which means it is a type of fiber that can promote the growth of beneficial bacteria in the gut. This can help to improve digestive health and support a diverse microbiome.

In further exemplary embodiments, the phenotype may have a beta-glucan content of approximately 20% to 25%, approximately 26% to 31%, approximately 32% to 37%, approximately 38% to 43%, and/or approximately 44% to 49%, as represented as a percentage of the total mycelium and sporocarps. The mycelium and sporocarps may be substantially dehydrated and in a powder form, a capsule form and/or in a drink mix powder for cold beverages application or a drink mix form for a hot beverage application.

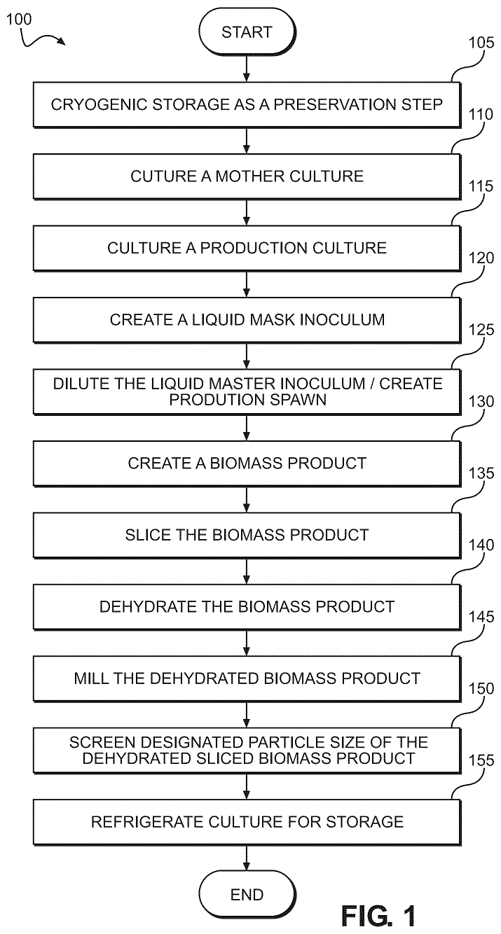

FIG. 1 is a flowchart 100 illustrating an exemplary method for propagating a Cordyceps militaris fungus, according to some embodiments of the present disclosure. At step 105, cryogenic storage is used as a preservation step. At step 110, the method may include culturing a mother culture. At step 115, the method may include culturing a production culture on a nutrient agar comprised of a mixture of dextrose, potato starch, nutritional yeast, and cauliflower powder. The production culture may be a sub-culture of the mother culture in various embodiments. At step 120, the method may include creating a liquid master inoculum using the production culture. At step 125, the method may include diluting the liquid master inoculum to create a diluted liquid master inoculum.

In some embodiments, at step 130, the method may include creating a biomass product using the diluted liquid master inoculum. At step 135, the method may include slicing the biomass product for dehydration to create a sliced biomass product. At step 140, the method may include dehydrating the sliced biomass product to create a dehydrated sliced biomass product. At step 145, the method may include milling the dehydrated sliced biomass product to select a designated particle size of the dehydrated sliced biomass product. At step 150, the method may include screening the designated particle size of the dehydrated sliced biomass product. At 155, a culture may be refrigerated for storage. More details according to various embodiments are disclosed below.

At step 110, a mother culture or preserved stock culture is cultured. According to exemplary embodiments, fresh cultures of the Cordyceps militaris mycelium of the strain designated M2-116-04 is cultured on nutrified agar and grown for a period of 7-10 days until a culture diameter of 30-40 mm is obtained. 5 mm×5 mm square sections of the healthiest part of the culture is cut from the leading edge of growth and placed into a 1.5 ml-2 ml cryogenic storage vial of 10-20% glycerol solution and cryogenically stored under Liquid Nitrogen (LN2). At the same time, 2 mm square sections of the same culture material is transferred to 10-20 culture tubes equal to or greater than 25 mm×150 mm in size and filled with a nutrient agar comprising a carbon/nitrogen/vitamin/mineral mix and subsequently stored under refrigeration between 34-40 degrees Fahrenheit (F). Culture tubes are sub-cultured every 6-12 months to new culture tubes to verify that the cultures are still active and healthy and to preserve the phenotypic qualities of the original cultures. Cryogenic cultures are recovered and sub-cultured into new culture tubes for storage and not used if any unwanted phenotypical variation is observed. Only growth representative of the mother culture is used for inoculum.

At step 115, a production culture is cultured. To start the production process, according to various exemplary embodiments, mother cultures are pulled from refrigeration and sub-cultured onto 4 nutrient agar plates. Cultures are allowed to grow for 9-16 days until a growth diameter of 30-40 mm is obtained. The healthiest and most representative tissue of the mother culture is sub-cultured at step 120.

At step 120, a liquid master inoculum is created, according to various embodiments. For example, nutrified liquid media is filled into 2 liter flasks and sterilized. They are then placed into a HEPA filtrated laminar flow hood to cool. Once cooled, a laboratory technician subcultures a 10 mm×10 mm section of the healthiest tissue from step 115 to each individual flask. The flasks are then stirred and incubated for 7-14 days under darkness and subsequently placed under refrigeration to prevent the culture from growing/fruiting further until use.

At step 125, the liquid master inoculum is diluted/create a production grain spawn to create a diluted liquid master inoculum, according to various embodiments. One day before biomass inoculation, a 10-liter carboy fitted with a port cap and a luer-lock dispensing system is sterilized with 8 liters of H₂0 and sterilized inside of an autoclave. The carboy is allowed to cool in front of a HEPA filtrated laminar flow hood. Once cooled, a laboratory technician dilutes the master liquid spawn into the 8 liters of H₂0 creating a 10-liter dilution solution of the inoculum. The inoculum is now ready for biomass inoculation.

At step 130, a biomass product is created, according to various embodiments. According to exemplary embodiments, 600 to 800 units of 8.5 pounds each of hydrated hulled oats, hydrated to a 50-60% moisture content in static and/or dynamic water tanks at 190-205 degrees F. for 30 min to 60 min is filled into an appropriate clear autoclavable bioreactor bag comprised of a polypropylene or high-density polyethylene or a polypropylene/high density polyethylene blend fitted with a microporous filter patch or strip material for gas exchange. The oats are subjected to sterilization in an autoclave for an appropriate time and temperature to fully render any present microorganisms inactive. The bags are cooled in a HEPA filtrated refrigerated cooldown room until the grain is equal to or less than 90 degrees F. Once cooled, the bags are delivered to a HEPA filtrated laminar flow hood and a laboratory technician transfers the dilution solution to the biomass bags at approximately 5 to 12.5 ml per bag. The bags are then incubated for approximately 35 to 55 days. The bags may comprise the sporocarps, mycelium and digested oats.

At step 135, the biomass product is sliced, according to various embodiments. Upon harvesting the biomass, in various exemplary embodiments, it is sliced though a dicer into smaller pieces and placed upon dehydration trays for dehydration.

At step 140, the biomass product is dehydrated, according to various embodiments. In various exemplary embodiments, the trays of biomass product are loaded into a static dehydrator and dried for approximately 24 hours and/or into a dynamic continuous dehydrator for approximately 30 to 60 minutes to a moisture content of <5%. This process is carefully watched to prevent altering the taste of the final product.

At step 145, the dehydrated biomass product is milled, according to various embodiments. The dried biomass is put though a mill and screened to a designated particle size and bulk packed for customers.

In some embodiments at step 150, the method may include screening the designated particle size of the dehydrated sliced biomass product. For example, the dried biomass is screened to a designated particle size and bulk packed for customers.

In some embodiments, at step 155, a culture may be refrigerated for storage.

In various embodiments, master culture storage parameters may include media additives such as yeast, rice protein, and cauliflower powder. Antibiotics including gentamicin and chloramphenicol may be used to ensure the culture is clean and free of contamination as a quality control measure. Subzero storage is acceptable, and storage may take place at 36 to 40 F.

In some embodiments, culturing parameters may include an incubation temperature of approximately 68 to 72 degrees F. in no or little light. Culturing times may include approximately 7 to 12 days with a 100 mm plate, approximately five days with a 25 mm×150 mm tube, and approximately 7 days with 500 ml of liquid. Culturing parameters may include media additives such as yeast, rice protein, and cauliflower powder.

In some embodiments, master spawn parameters may include an incubation temperature of approximately 68 to 72 degrees F. in no or little light. Culturing time may be approximately 7 to 14 days with liquid culture. Culturing parameters may include media additives such as yeast, rice protein, and cauliflower powder. Storage may take place at 36 to 40 degrees F.

In some embodiments, production spawn parameters may include media of oats and sorghum with a large bioreactor (8.5 pounds) for approximately 10 days in little or no light. The culture may be shaken on approximately day 6. Storage may take place at 36 to 40 degrees F.

In some embodiments, biomass product parameters may include oats as media, incubation temperature of approximately 68 to 72 F, with approximately 54 days in a large bioreactor (8.5 pounds). Light is provided at approximately 12 hours on and 12 hours off. The 12 hours of on light intensity may be at an intensity of 300 to 500 lux, wavelength blue at approximately 450 nm plus red at 650 nm.

In various exemplary embodiments, a peristaltic pump may be used for dispensing liquid inoculum into bioreactor bags filled with grain media. A bioreactor bag is an autoclavable HDPE, PP or HDPE/PP blend bag equipped with membrane air filter to allow mushroom culture to breathe during the incubation period. Inoculation is a key step in the production process and inattention to procedures and/or aseptic technique can result in microbial contamination and the loss of the entire batch.

In some embodiments, hydration of organic grain (substrate) may take place in static hydration tanks and/or dynamic hydration tanks prior to the semi-automated bag filling and sterilization operations. It is typically a first step in the processing of the substrate used for the solid-state fermentation of mycelial biomass for Cordyceps militaris.

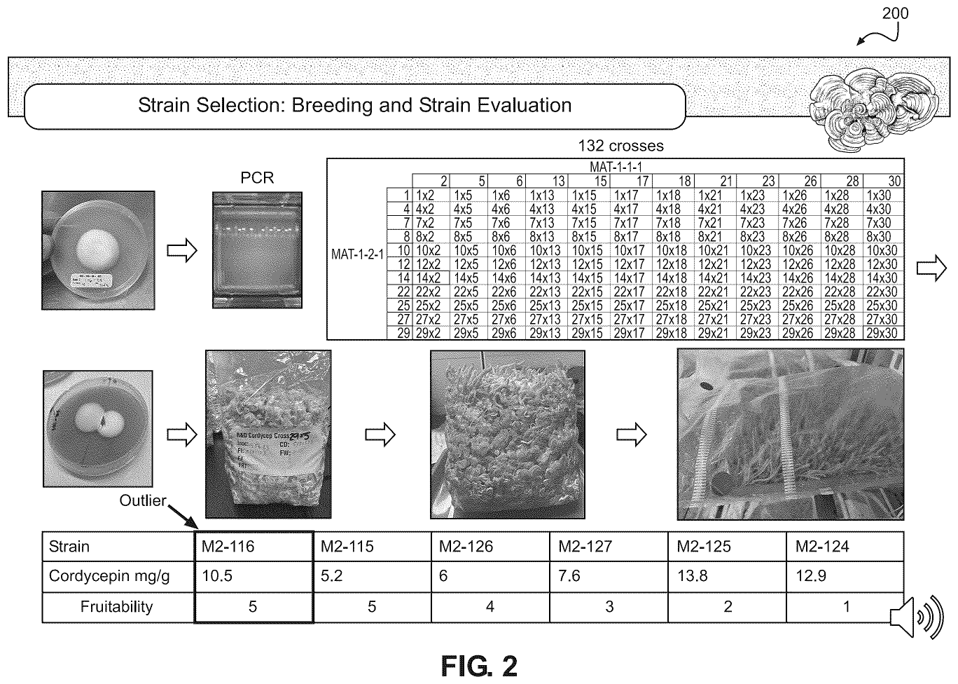

In some embodiments, breeding selection of the improved, not naturally occurring Cordyceps militaris designated as strain M2-116-04. For example, see FIG. 2. FIG. 2 is a drawing 200 of an overview of the selection by breeding and strain selection to produce the improved, not naturally occurring Cordyceps militaris designated as strain M2-116-04, according to some embodiments of the present disclosure. Significantly, Cordyceps militaris fungus designated as strain M2-116-04 has markedly different characteristics than naturally occurring strains including different functional and structural characteristics that rise to the level of a marked difference. For example, Cordyceps militaris fungus designated as strain M2-116-04 is not naturally occurring and is the product of human intervention that includes increased cordycepin (C) content in the sporocarp and biomass compared with other strains.

According to some embodiments, spores of an Cordyceps militaris strain of M2-105-04 are collected from in house grow operation. This is done in a sterile environment by taping stroma with fully developed perithecia on the lid of an empty 100 mm petri dish and allowing the spores to fall to the lower part of the petri dish.

Spores are then scrapped lightly from the deposited spores and placed in suspension in sterile water.

Serial dilutions are made to a factor of 1:1000 and plated to Potato/dextrose/cauliflower agar with 100 μL per plate.

Single spore isolates are collected from these plates and grown out to the size of a 2 cm. Each single spore isolates culture is given a series number. 1, 2, 3, 4 and so forth.

Each plate is replicated by a factor of 10 plates to a growth diameter of 2 cm and put into cold storage.

Each isolate backed up into 2 ml cryogenic vials and frozen under LN2 to be preserved and to allow recovery for a later date if needed.

DNA is extracted and purified of each isolate.

PCR is performed with the following primers to categorize them by MAT 1 and MAT 2, their mating types determining their ability to mate.

MAT1-1-1 Forward primer.
5′-ATGGAACAC AGATCGAGCGACAC-3′
MAT 1-1-1 Reverse primer.
5′-ATATACCTTCGC GATCATTGCCCAG-3′
MAT 1-2-1 Forward primer.
5′-TGTTTTGTCGCGATG GTTCTGG-3′
MAT 1-2-1 Reverse primer.
5′-CCTCTGGAGGTTCTGCAT TCCA-3′

All possible crosses are determined, and each isolate is paired with a potential mate.

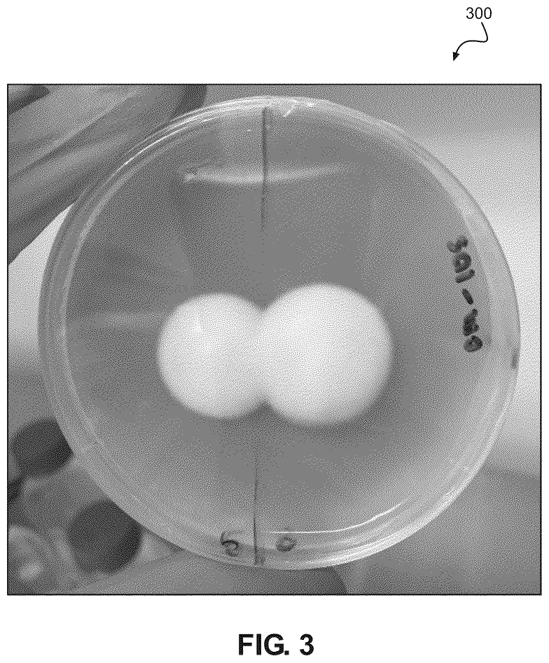

Each pair is plated to agar with a small distance between them. Usually a distance of 1-2 cm. For example, See FIG. 3. FIG. 3 is a drawing 300 of an agar plate culturing Cordyceps militaris for selecting improved, not naturally occurring strains with markedly different characteristics than naturally occurring strains including different functional and structural characteristics that rise to the level of a marked difference, according to various embodiments of the present disclosure.

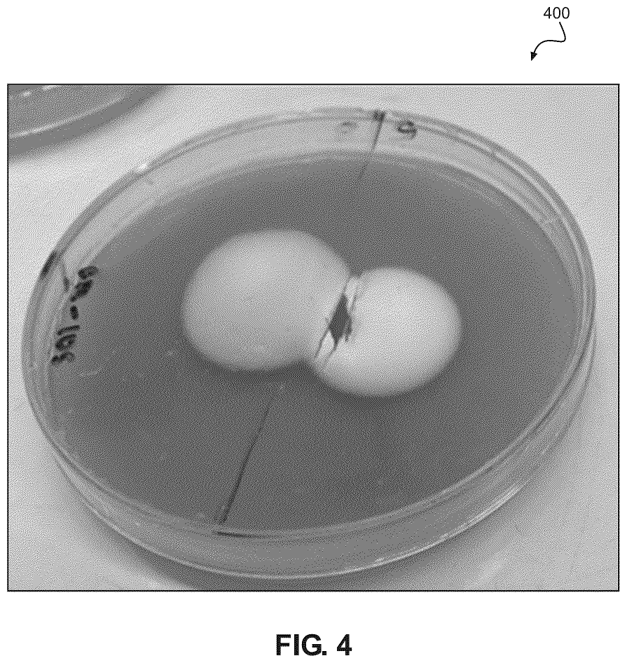

Once the mating occurs a small section of the overlapping mycelium is cut from the growing culture and re-plated and assigned a temporary name until each culture is evaluated for its fruiting potential. For example, See FIG. 4. FIG. 4 is a drawing 400 of an agar plate culturing Cordyceps militaris for selecting improved, not naturally occurring strains, including a small section of the overlapping mycelium cut, according to various embodiments of the present disclosure.

Each new cross is replicated ten times and backed up into cryovials, frozen in LN2 and saved for future recovery if needed.

50 ml black centrifuge vials are filled with 25 ml of nutrified liquid media consisting of Potato starch, dextrose, nutritional yeast, cauliflower powder and H2O. Three tubes are made for each paired culture. These centrifuge tubes are sterilized in an autoclave and cooled in front of a laminar flow hood.

Three 50 ml tubes are inoculated with each new paired culture and rotated on a lab rotator for 5 days.

A one pound bioreactor bag of hydrated oats, hydrated to 55-58% moisture content are sterilized inside an autoclave and cooled inside a laminar flow hood. Two bags for each paired culture.

Two 50 ml tubes of each set are aseptically poured into two qty 1-pound bags of hydrated sterilized oats in breathable bioreactor bags and shaken to disperse the inoculum.

Each bag is grown for a period of 50-65 days and evaluated for the following parameters and recorded, as listed below.

Days to full consolidation of the bag with the organism.

Days till pigmentation.

Intensity of pigmentation.

Days to sporocarp initiation.

Days to full sporocarp development.

Coverage of sporocarps.

Sexual viability. Ability to complete its sexual life cycle.

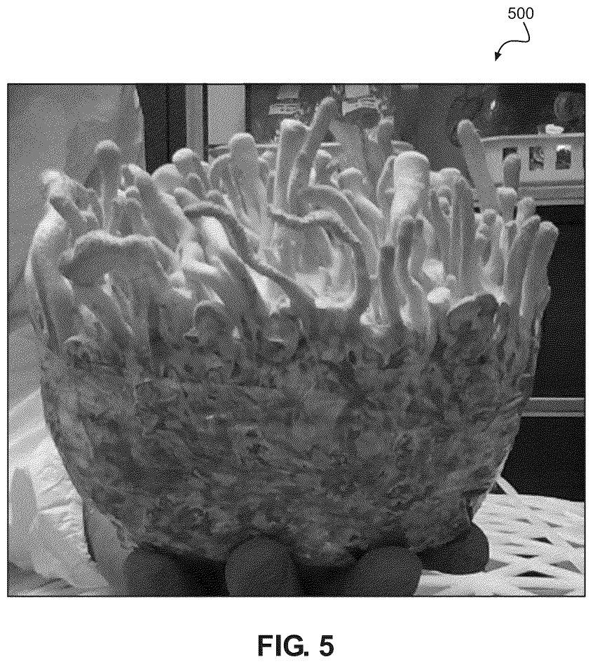

For example, see FIG. 5. FIG. 5 is a color drawing 500 of a fully colonized and fully fruited one pound block of not naturally occurring Cordyceps militaris fungus designated as strain M2-116-04 that has markedly different structural characteristics that rise to the level of a marked difference than naturally occurring stains, according to various embodiments of the present disclosure.

Each culture exhibiting the best fruiting traits is then labelled with new official strain designations. For example, naming may proceed as the following strains: Strain M2-115-04, strain M2-116-04, strain M2-117-04, strain M2-118-04, strain M2-119-04, strain M2-120-04, strain M2-121-04, strain M2-122-04, strain M2-123-04, Strain M2-124-04, strain M2-125-04, strain M2-126-04, strain M2-127-04, and so forth.

Sporocarps and mycelial biomass for each new selected strain is tested for Cordycepin levels and fungal beta glucans.

The best six of these strains Cordyceps militaris (e.g., Strain M2-115-04, strain M2-116-04, Strain M2-124-04, strain M2-125-04, strain M2-126-04, strain M2-127-04) as evaluated by the criteria above (i.e., criteria including sporocarps and mycelial biomass for cordycepin levels and fungal beta glucans), which are then grown out in 8.5 pound bags of hydrated sterilized oats in breathable bio reactors bags to simulate a production run.

The bags were then evaluated during their growth in the same manner as presented above to ensure consistency of the growth process.

While all strains produced sporocarps and coloration as they did in the 1-pound bag evaluation, the not naturally occurring Cordyceps militaris fungus designated as strain M2-116-04 was very consistent and reproducible in its characteristics and was selected as a premium commercial strain as it was both capable of producing a high coverage of sporocarps and still able to maintain high amounts of Cordycepin in both the mycelial biomass and the sporocarps, which are markedly different functional characteristics that rise to the level of a marked difference than naturally occurring stains. The not naturally occurring Cordyceps militaris fungus designated as strain M2-116-04 was further evaluated for antioxidant capacity under cell studies and run under time-of-flight mass spectrometry to determine if Cordyceps militaris fungus designated as strain M2-116-04 had any significant amounts of other desirable compounds within its structure and to compare it to other mushroom product on the US market.

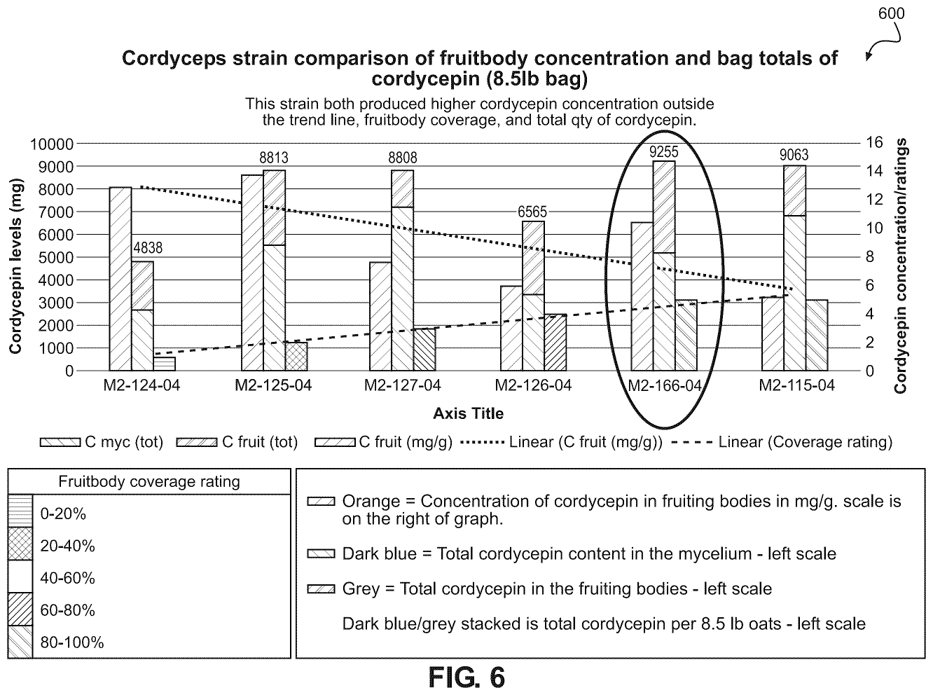

For example, see FIG. 6. FIG. 6 is a table 600 showing the trending of cordycepin (C) content in the sporocarps (e.g., fruiting bodies) and biomass ordered by linear coverage of the sporocarps (e.g., fruits) for various strains of Cordyceps militaris fungus including the not naturally occurring strain M2-116-04, according to various embodiments of the present disclosure. For example, FIG. 6 shows cordycepin (C) content in sporocarps and biomass ordered by linear coverage of sporocarps for strains of Cordyceps militaris (e.g., Strain M2-115-04, strain M2-116-04, Strain M2-124-04, Strain M2-125-04, Strain M2-126-04, and Strain M2-127-04). Significantly, the not naturally occurring Cordyceps militaris fungus designated as strain M2-116-04 has markedly different functional characteristics that rise to the level of a marked difference than naturally occurring stains including higher total cordycepin concentration.

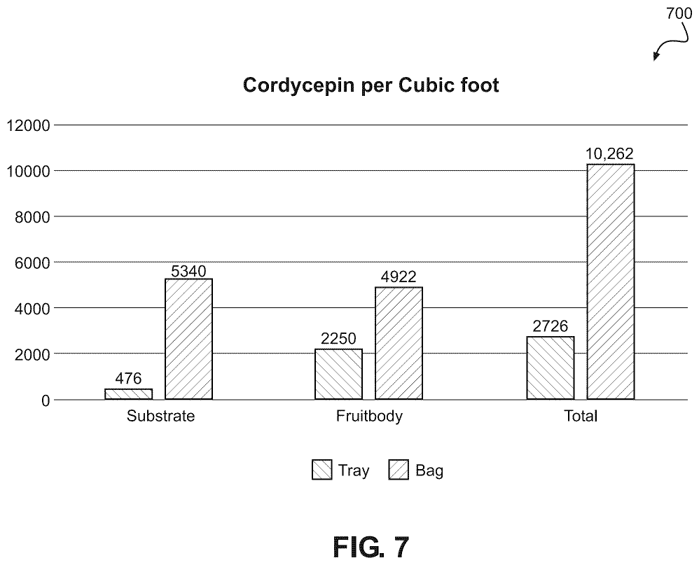

Cordycepin per square foot advantage. For example, see FIG. 7. FIG. 7 is a table 700 showing cordycepin in Cordyceps militaris per cubic foot of grow space using tray culture compared with liquid bag culture, according to various embodiment of the present disclosure.

Significantly, the not naturally occurring Cordyceps militaris fungus designated as strain M2-116-04 has markedly different functional characteristics that rise to the level of a marked difference than naturally occurring stains including higher total cordycepin concentration. For example, Cordyceps militaris fungus designated as not naturally occurring strain M2-116-04 produced the highest coverage of sporocarps on the surface of the block while still maintaining elevated levels of cordycepin overall within the biomass and sporocarps. Cordycepin and many precursor components are processed in the mycelium and concentrated in the sporocarps of Cordyceps militaris near peak fruiting. Mycelial biomass of most strains of Cordyceps militaris comprises cordycepin amounts in the range of 0.5 to 2 mg/g even at this peak moment in fruiting. This makes it a valuable component to include in a product. The present technology determined that using the systems and methods described herein produced 276% more cordycepin per cubic foot of grow space when it came to the total biomass plus sporocarps using liquid bag culture using Cordyceps militaris fungus designated as not naturally occurring strain M2-116-04. Up to 25 pounds of biomass may fit within the same square footage one tray takes up. Tray culture, the most common way of producing sporocarps, utilizes thin substrates and high biological efficiencies (the ratio of sporocarps in weight to the substrate it is grown on) to produce the maximum amount of sporocarps. Using tray culture, the sporocarps are harvested and the substrate is discarded and composted. Most strains in the current industry are bred for growing in tray culture and are usually grown on brown rice with some form of supplementation. Even though there is a higher number of sporocarps produced in tray culture, the lack of mycelial biomass infrastructure holds back the concentration of cordycepin within them. The present technology uses liquid culture and strains bred and evaluated on larger substrates allow the sporocarps to concentrate the cordycepin with higher potential values due to having the infrastructure in the mycelial network to support it. The present technology evaluated six potential in house strains (e.g., Strain M2-115-04, Strain M2-116-04, Strain M2-124-04, Strain M2-125-04, Strain M2-126-04, and Strain M2-127-04) during our breeding project that showed that one not naturally occurring strain of Cordyceps militaris fungus designated as strain M2-116-04 was able to overcome these trends. Cordyceps militaris fungus designated as not naturally occurring strain M2-116-04 produced the highest coverage of sporocarps on the surface of the block while still maintaining elevated levels of cordycepin overall within the biomass and sporocarps.

According to various embodiments, average cordycepin content of sporocarps between tray culture (industry standard) and liquid bag culture of the present technology. For example, see FIG. 7. FIG. 7 is a table 700 showing Cordycepin in Cordyceps militaris fungus designated as the not naturally occurring strain M2-116-04 per cubic foot of grow space using tray culture compared with liquid bag culture including room parameters, according to various embodiment of the present disclosure.

Tray culture: Used by most growers (thin 1-1.5 inch thick substrate). For example, current industry (avg 3.7) 1.4-6 mg/g (Peak 9 mg/g).

Liquid Bag culture (present technology) 8.5 lb. block of grain 9″×6″×8″ (L×W×H). For example, (avg 10.7) 7.6-13.8 mg/g (peak 17.2 mg/g).

Average Cordycepin content of mycelium between tray culture (industry standard) and liquid bag culture of the present technology.

Tray culture: Used by most growers (thin 1-1.5 inch thick substrate). For example, current industry (avg 0.75 mg/g) 0.6-0.9 mg/g (Peak 1.4 mg/g).

Liquid Bag culture (present technology) 8.5 lb. block of grain 9″×6″×8″ (L×W×H). For example, (avg 1.25 mg/g) 0.5-2 mg/g (peak 2.4 mg/g).

Cordycepin concentration of Cordyceps militaris fungus designated as the not naturally occurring strain M2-116-04 sporocarps using liquid bag culture of the present technology is 10.50 mg/g.

Cordycepin concentration of Cordyceps militaris fungus designated as the not naturally occurring strain M2-116-04 mycelium using liquid bag culture of the present technology is 1.53 mg/g.

Phenotype description of Cordyceps militaris fungus designated as the not naturally occurring strain M2-116-04 as follows with markedly different characteristics than naturally occurring strains.

Microscopic characterization was performed on cultures, fresh and dried collections using diagnostic characters included in taxonomic treatments (See Kobayasi Y. 1941. The genus Cordyceps and its allies, Science reports of the Tokyo Daigaku. 84:53-260; Mains E B. 1958. North American entomogeneous fungi. Bull Torrey Bot Club. 78 (2): 122-33; and Sung G H, Hywel-Jones N L, Sung J M, Luangsa-ard J J, Shrestha B, Spatafora W. 2007. Phylogenetic classification of Cordyceps and the Clavicipitaceous fungi. Stud Mycol. 57:5-59.

The dried samples were rehydrated for 5 minutes in isopropyl alcohol and stained for 5 minutes with cresyl blue. Stroma length was measured in both the fertile part and the stipe, the perithecium width and length, ascus width and length, apical cap, ascospore width, and part-spore length and width. The cultures' vegetative hyphal width, conidiophores, phialides were also measured. (Images were taken with a Euromex iScope compound light microscope using AmLite HD408 4K 60 fps 8MP HDMI, LAN-Wi-Fi Color CMOS C-mount Microscope Camera for PC Imaging.)

ASCOCARP-Stroma (57.5) 39-79 mm tall×(4.5) 2-9 mm wide at the apex and (4.7) 3.5-4 mm wide where attached. Stipe clavate to subclavate, can be tapered to equal. Apex is often bulbous, always with a rounded terminus. Stipitipellis cartilaginous when fresh to fleshy-fibrous with drying. Most often with a basal tomentum. Pith sometimes chambered, mostly solid and cottony.

Exoperidium tissue up to 0.1 mm thick will peel when pulled. Endoperidium 2 mm thick, cartilaginous, and solid to flexible. Stroma (5A6-5A8) melon yellow to chrome yellow, staining when bruised to (6B8) tangerine-orange peel. Pith (4A1-4A3) white to pale yellow-cream, oxidizing when fresh cut (5B5-5E6) light orange to yellowish brown-mustard brown, sometimes to (6F8) brownish grey at the base of the stroma. Stroma peridium comprised of thin-walled globose to irregular shaped hyaline cells of smooth ornamentation, entangled with mycelia strands and club-shaped septate cells. Stroma tissue plectenchyma-like and readily staining with cresyl blue.

Perithecia (84) 73-97 μm long×(56.5) 33-56 μm wide are partially immersed in the stipitipellis on the stroma. Perithecia pyriforme to subpyriforme comprised of long mycelial threads with blunt ended terminus and rounded edges, non-septate, thin-walled hyaline, mostly non-branching with rare dichotomous branching. Having pigmented cystidia-like hyphal threads radiating out of the perithecia. Cystidia thin walled, septate, (0.45) 0.4-0.6 μm long. Thin-walled hyphal tissue readily staining green-blue with cresyl blue under compound light microscopy. Operculum (10) 5-14.8 μm broad.

ASCOSPORES—disarticulating ascospores erupting from long cylindrical asci (up to 500-800 μm in length). Colorless, filiform, with numerous partitions equal in length, breaking down into individual cylindrical cells with maturity, smooth, non-pigmented. Developing asci with filiform ascospores disarticulating into part-spores. Ascospores are thread-like, colorless, and segmented, (4) 3.5-6 μm long×(1) 1-1.5 μm wide, which then germinate to produce hyphal strands and secondary conidia. Ascus cap (1.5) 1.2-2.3 μm tall×(3.2) 2.8-3.3 μm wide. Ascus hyphal threads (3.5) 3.2-3.9 μm wide. Conidiospores (1.8) 1.1-2.7 μm broad×(2.3) 1.5-2.8 μm wide.

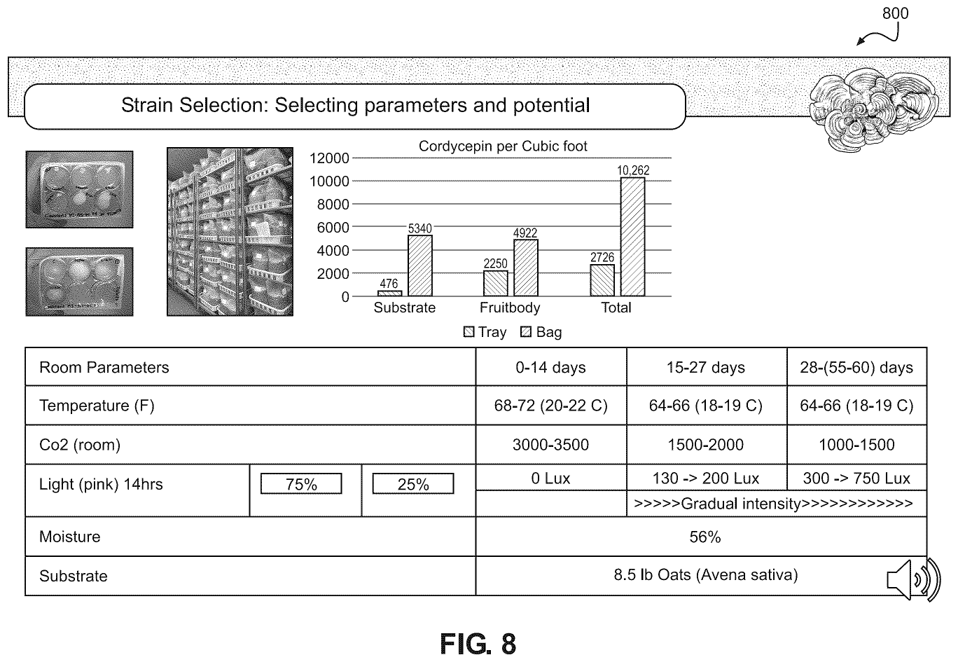

FIG. 8 is a drawing 800 of an overview of the selection by breeding and strain selection including parameters and potential to produce the improved, not naturally occurring Cordyceps militaris designated as strain M2-116-04, according to some embodiments of the present disclosure.

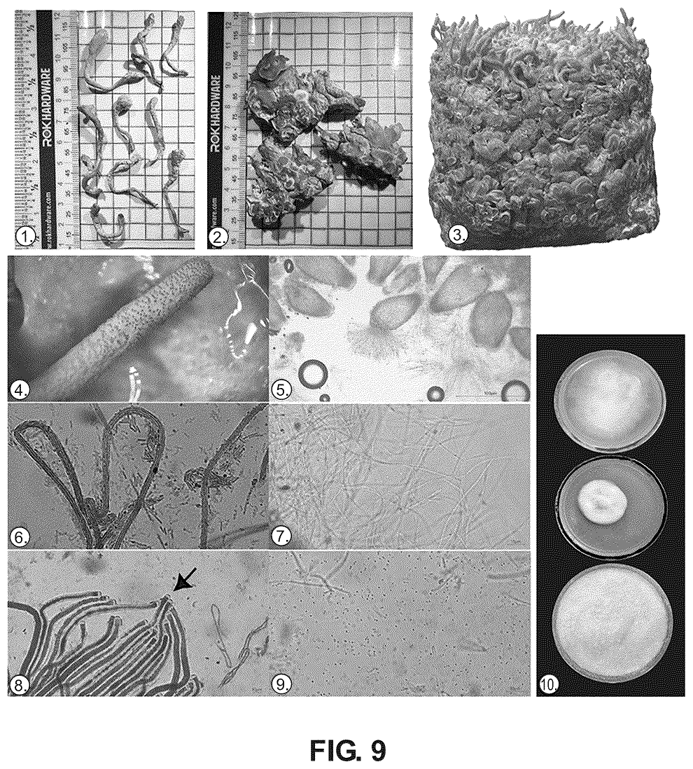

FIG. 9 are color drawings 900 showing a phenotype description of Cordyceps militaris fungus designated as the not naturally occurring strain M2-116-04, according to embodiments of the present disclosure. For example, the phenotype description of Cordyceps militaris fungus designated as the not naturally occurring strain M2-116-04 shown in FIG. 9 includes sporocarps and microscopic morphology in [1.], dried stroma in [2.], developed sclerotium grown along bioreactor bag walls in [3.], fresh stroma and sclerotium growing on grain in [4.], fresh stroma with perithecia. [5.], perithecia with asci erupting from within at 40× with light microscopy in [6.], Asci with ascospores and part-spores at 100× with light microscopy in [7.], and mycelial strands of stroma at 40× with light microscopy in [8.], Asci with ascus caps at 100× with light microscopy in [9.] Conidiospores at 40× with light microscopy Cordyceps militaris growing on potato dextrose agar+cauliflower (PDAC) petri plates. Top plate: fully colonized in the dark. Middle plate: 6-day old culture. Bottom plate: Liquid culture quality control plate on tryptic soy agar (TSA) in [10.] (see López-Rodríguez, L., Burrola-Aguilar, C., Garibay-Orijel, R., Estrada-Zúñiga, M. E., Matías-Ferrer, N., & Argüelles-Moyao, A. (2022). Cordyceps mexicana sp. nov., parasitizing Paradirphia sp. moths: A new sister species of the Cordyceps militaris complex, distributed in central Mexican Quercus-Pinus mixed forests. Mycologia, 114 (4), 732-747.

According to some embodiments, a not naturally occurring Cordyceps militaris fungus designated as strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802.

According to some embodiments, a not naturally occurring Cordyceps militaris fungus designated as strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802.

According to some embodiments, the not naturally occurring Cordyceps militaris fungus designated as strain M2-116-04 characterized by a phenotype having an abundance of sporocarps on top of a substrate and along a parameter of a bioreactor bag.

According to some embodiments, the not naturally occurring Cordyceps militaris fungus designated as strain M2-116-04 characterized by a phenotype having fruiting initiation at or near 18 days after inoculation.

According to some embodiments, the not naturally occurring Cordyceps militaris fungus designated as strain M2-116-04 characterized by a phenotype having sporocarps maturing at or near 55 days after inoculation.

According to some embodiments, the not naturally occurring Cordyceps militaris fungus designated as strain M2-116-04 characterized by a phenotype having a 10-to-15-day colonization time on hulled oats.

According to some embodiments, the not naturally occurring Cordyceps militaris fungus designated as strain M2-116-04 characterized by a phenotype optimized to grow on oats.

According to some embodiments, the not naturally occurring Cordyceps militaris fungus designated as strain M2-116-04 characterized by a phenotype having a beta-glucan content of approximately 20% to 25% as represented as a percentage of a total mycelium and sporocarps.

According to some embodiments, the not naturally occurring Cordyceps militaris fungus designated as strain M2-116-04, characterized by a phenotype having a beta-glucan content of approximately 26% to 31% as represented as a percentage of a total mycelium and sporocarps.

According to some embodiments, the not naturally occurring Cordyceps militaris fungus designated as strain M2-116-04 characterized by a phenotype having a beta-glucan content of approximately 32% to 37% as represented as a percentage of a total mycelium and sporocarps.

According to some embodiments, the not naturally occurring Cordyceps militaris fungus designated as strain M2-116-04 characterized by a phenotype having a beta-glucan content of approximately 38% to 43% as represented as a percentage of a total mycelium and sporocarps.

According to some embodiments, the not naturally occurring Cordyceps militaris fungus designated as strain M2-116-04 characterized by a phenotype having a beta-glucan content of approximately 44% to 49% as represented as a percentage of a total mycelium and sporocarps.

According to some embodiments, the not naturally occurring Cordyceps militaris fungus designated as strain M2-116-04, characterized by a phenotype having a cordycepin content of approximately 0.4 milligrams per gram to 1.1 milligrams per gram as represented relative to a total mycelium and sporocarps.

According to some embodiments, the not naturally occurring Cordyceps militaris fungus designated as strain M2-116-04 in a powder form.

According to some embodiments, the not naturally occurring Cordyceps militaris fungus designated as strain M2-116-04 in a capsule form.

According to some embodiments, the not naturally occurring Cordyceps militaris fungus designated as strain M2-116-04 in a drink mix form for cold and hot beverages applications.

According to some embodiments a method for treating a mammal to improve immune functioning of the mammal, the method comprising administering an effective dose of Cordyceps militaris mushroom powder comprising Cordyceps militaris mycelium and sporocarps.

According to various embodiments, Cordyceps militaris fungus designated as strain M2-116-04 is improves immune functioning as shown by Exhibit 1 and Exhibit 2. Exhibit 1 is a manuscript for Journal of Functional Foods, “Prebiotic activity of functional whole mushroom powders in short-term in vitro colonic simulations” (First Author: Julie Daoust), which is incorporated by reference herein for all purposes. Exhibit 2 is a manuscript for-ProDigest Gastrointestinal Expertise, “A Screening SHIME® Study to Rank Prebiotic Activity of Functional Mushrooms” which is incorporated by reference herein for all purposes.

According to some embodiments wherein the effective dose is administered once every 12 days over 8 separate consecutive 12-day intervals.

According to some embodiments, wherein the Cordyceps militaris mushroom powder comprises not naturally occurring Cordyceps militaris fungus designated as strain M2-116-04.

According to some embodiments further comprising the effective dose being 500 mg of the Cordyceps militaris mushroom powder per kg of body weight of the mammal.

According to some embodiments further comprising the effective dose being 1000 mg of the Cordyceps militaris mushroom powder per kg of body weight of the mammal.

According to some embodiments comprising the effective dose being 2000 mg of the Cordyceps militaris mushroom powder per kg of body weight of the mammal.

According to some embodiments further comprising the mammal being a human.

According to some embodiments a method for treating a mammal to improve gut functioning of the mammal, the method comprising administering an effective dose of Cordyceps militaris mushroom powder comprising not naturally occurring Cordyceps militaris fungus designated as strain M2-116-04, the mushroom powder comprising Cordyceps militaris mycelium and sporocarps and being a prebiotic.

Cordyceps militaris mushroom powder comprising not naturally occurring Cordyceps militaris fungus designated as strain M2-116-04, the mushroom powder comprising Cordyceps militaris mycelium and sporocarps and being a prebiotic

According to various embodiments, Cordyceps militaris fungus designated as strain M2-116-04 is a prebiotic as shown by Exhibit 1 and Exhibit 2. Exhibit 1 is a manuscript for Journal of Functional Foods, “Prebiotic activity of functional whole mushroom powders in short-term in vitro colonic simulations” (First Author: Julie Daoust), which is incorporated by reference herein for all purposes. Minimally processed, whole mushroom powders including Cordyceps [Cordyceps militaris], were evaluated for their effects on the human gut microbiota. Short-term in vitro colonic simulations were performed. Metabolic changes, including significant increases in acetate, propionate, and butyrate levels were observed with all test products versus untreated control. Further, beneficial bacteria were enhanced with all test products. Caco-2/THP1 co-culture studies with fermentations from stains including Cordyceps demonstrated significant improvement in intestinal barrier function with all test products versus untreated control (fecal donor-dependent). Test product-dependent/donor-dependent pro-inflammatory molecule (TNFα, CXCL10, MCP-1) decreases and anti-inflammatory cytokine (IL-6 and IL-10) increases were observed versus untreated control. Tryptamine levels increased and pyruvic acid levels decreased with these test products versus untreated control. All six mushroom products including Cordyceps militaris demonstrated prebiotic properties.

According to various embodiments, Cordyceps militaris fungus designated as strain M2-116-04 is a prebiotic as shown by Exhibit 1 and Exbibit 2. Exhibit 2 is a manuscript for-ProDigest Gastrointestinal Expertise, “A Screening SHIME® Study to Rank Prebiotic Activity of Functional Mushrooms” which is incorporated by reference herein for all purposes. The aim of the study was the assessment of the effect of three functional mushroom formulas on the gut microbiota and composition and to compare this to a positive control, i.e., inulin. Interindividual variability of the gut microbiota were considered by evaluating three healthy adults. The experiment to mechanistically unravel the microbial modulation by the mushroom formulas was conducted using the screening SHIME® technology. Significantly increased base-acid consumption was observed for each of the mushroom formulas across donors during the first week of treatment and a trend towards enhanced acidification in the following days. These results indicate enhanced fermentation activity. Significant increases in gas production over the whole 48 hour incubation were observed across donors for the positive control, Digestive Blend and Cordyceps. Most gas production occurred within the first 24 hours of incubation for all products, albeit to a slightly lesser extent for the positive control. The final 24 hours were characterized by increased gas production for the positive control as compared to the test products. During the first week of treatment, acetate production in the negative and positive control was similar, while there was a trend towards enriched acetate levels for the mushroom formulas. Indeed, acetate production was significantly increased across donors compared to the positive control for Cordyceps on day 3 and on day 8 for MaxSpectrum Blend. Interindividual responses became more pronounced during the second week of the study. Propionate production tended to be reduced upon treatment, although low consistency was observed across donors. A single dose of the Digestive Blend resulted in significantly increased propionate levels across donors but this effect did not continue in the following days. Butyrate production was enriched by all treatments across donors. While for the positive control, butyrate levels were consistently enhanced over time compared to the negative control, for the mushroom formulas, there was a gradual increase over time. Indeed, on day 3 and 8, butyrate production was significantly boosted for Cordyceps compared to the positive control. Moreover, on the final days of the study there was a strong trend towards increased butyrate levels for all mushroom formulas compared to the positive control. Lactate levels remained low throughout the complete experimental period for all donors, indicating the establishment of cross-feeding interactions towards propionate and/or butyrate. Finally, with regards to proteolytic markers, there was a trend towards increased NH4-N levels across donors, mostly observed on day 5. This increase in NH4-N is to be expected due to the protein content of each of the test products. bCFA levels were generally low for all conditions and only limited effects in terms of production were induced across donors upon treatment. Shallow shotgun sequencing confirmed changes in metabolic activity induced by the different products, with the strongest effects on microbial community composition observed on day 12. Beta-diversity analysis revealed that the positive control inulin exhibited a less pronounced shift from the negative control compared to the test-products, which suggests a smaller impact on gut microbiota. In contrast, Digestive Blend, Cordyceps, and MaxSpectrum Blend each demonstrated large impacts on the gut microbial community, outperforming inulin. Specifically, growth of beneficial bacteria such as Bifidobacterium was stimulated following Cordyceps treatment as compared to the negative control, while all test products stimulated growth of various butyrate-producing groups from the Lachnospiraceae and Ruminococcaceae families in both luminal and mucosal gut environment, which aligns with the observed butyrogenic effects. Furthermore, acetate-producing, gas-consuming Blautia_A spp. may have benefited from the enhanced gas production upon fermentation of the test products. Lastly, Cordyceps treatment did not stimulate growth of opportunistic pathogenic species Pseudomonas aeruginosa as compared to the other test products. Upon performing LA-REIMS metabolic fingerprinting, a total of 1555 metabolic features was detected in the set of in vitro fluids from the SHIME® experiment. Based on these fingerprints, significant metabolic shifts could be revealed between the positive control (inulin) and each of the different mushroom treatment conditions as well as the negative control. Overall, the various products showed a relatively high level of similarity in their effects. However, significant segregation could be observed for the MaxSpectrum Blend, mainly resulting from a higher responsiveness of donor B for this treatment compared to the Digestive Blend and Cordyceps treatment. Evaluation of the treatment duration showed that a prolonged administration of at least 8 days was needed to, in general, have a clear onset of the treatment effects.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the technology as defined by the appended claims. Thus, the breadth and scope of a preferred embodiment should not be limited by any of the above-described exemplary embodiments.

What is claimed is:

1. A not naturally occurring Cordyceps militaris fungus designated as strain M2-116-04 as deposited with the ATCC Patent Depository under the Budapest Treaty (10801 University Blvd, Manassas, VA 20110), on Jul. 11, 2024, with the unofficial ATCC Patent Deposit Designation No. PTA-127802, and the official deposit date of Jul. 11, 2024, and the official Patent Deposit Designation No. PTA-127802.

2. The not naturally occurring Cordyceps militaris fungus designated as strain M2-116-04 of claim 1, characterized by a phenotype having an abundance of sporocarps on top of a substrate and along a parameter of a bioreactor bag.

3. The not naturally occurring Cordyceps militaris fungus designated as strain M2-116-04 of claim 1, characterized by a phenotype having fruiting initiation at or near 18 days after inoculation.

4. The not naturally occurring Cordyceps militaris fungus designated as strain M2-116-04 of claim 1, characterized by a phenotype having sporocarps maturing at or near 55 days after inoculation.

5. The not naturally occurring Cordyceps militaris fungus designated as strain M2-116-04 of claim 1, characterized by a phenotype having a 10-to-15-day colonization time on hulled oats.

6. The not naturally occurring Cordyceps militaris fungus designated as strain M2-116-04 of claim 1, characterized by a phenotype optimized to grow on oats.

7. The not naturally occurring Cordyceps militaris fungus designated as strain M2-116-04 of claim 1, characterized by a phenotype having a beta-glucan content of approximately 20% to 25% as represented as a percentage of a total mycelium and sporocarps.

8. The not naturally occurring Cordyceps militaris fungus designated as strain M2-116-04 of claim 1, characterized by a phenotype having a beta-glucan content of approximately 26% to 31% as represented as a percentage of a total mycelium and sporocarps.

9. The not naturally occurring Cordyceps militaris fungus designated as strain M2-116-04 of claim 1, characterized by a phenotype having a beta-glucan content of approximately 32% to 37% as represented as a percentage of a total mycelium and sporocarps.

10. The not naturally occurring Cordyceps militaris fungus designated as strain M2-116-04 of claim 1, characterized by a phenotype having a beta-glucan content of approximately 38% to 43% as represented as a percentage of a total mycelium and sporocarps.

11. The not naturally occurring Cordyceps militaris fungus designated as strain M2-116-04 of claim 1, characterized by a phenotype having a beta-glucan content of approximately 44% to 49% as represented as a percentage of a total mycelium and sporocarps.

12. The not naturally occurring Cordyceps militaris fungus designated as strain M2-116-04 of claim 1, characterized by a phenotype having a cordycepin content of approximately 0.4 milligrams per gram to 1.1 milligrams per gram as represented relative to a total mycelium and sporocarps.

13. The not naturally occurring Cordyceps militaris fungus designated as strain M2-116-04 of claim 1, in a powder form.

14. The not naturally occurring Cordyceps militaris fungus designated as strain M2-116-04 of claim 1, in a capsule form.

15. The not naturally occurring Cordyceps militaris fungus designated as strain M2-116-04 of claim 1, in a drink mix form for cold and hot beverages applications.

16. A method for treating a mammal to improve immune functioning of the mammal, the method comprising administering an effective dose of Cordyceps militaris mushroom powder comprising Cordyceps militaris mycelium and sporocarps.

17. The method of claim 16, wherein the effective dose is administered once every 12 days over 8 separate consecutive 12-day intervals.

18. The method of claim 16, wherein the Cordyceps militaris mushroom powder comprises not naturally occurring Cordyceps militaris fungus designated as strain M2-116-04.

19. The method of claim 16, further comprising the effective dose being 500 mg of the Cordyceps militaris mushroom powder per kg of body weight of the mammal.

20. The method of claim 16, further comprising the effective dose being 1000 mg of the Cordyceps militaris mushroom powder per kg of body weight of the mammal.

21. The method of claim 16, further comprising the effective dose being 2000 mg of the Cordyceps militaris mushroom powder per kg of body weight of the mammal.

22. The method of claim 16, further comprising the mammal being a human.

23. A method for treating a mammal to improve gut functioning of the mammal, the method comprising administering an effective dose of Cordyceps militaris mushroom powder comprising not naturally occurring Cordyceps militaris fungus designated as strain M2-116-04, the mushroom powder comprising Cordyceps militaris mycelium and sporocarps and being a prebiotic.
